(19) United States
(12) Patent Application Publication
(10) Pub. No.: US 2023/0060321 A1
(43) Pub. Date: Mar. 2, 2023

(54) AUTOMATIC DETECTION TRIGGER DEVICE FOR QUANTITATIVE COMPRESSED ADMINISTRATION AEROSOL

(71) Applicant: Shanghai Sonmol Internet Technology Co., Ltd., Shanghai City (CN)

(72) Inventors: ZhengHao Ye, HeNan Province (CN); JiGuang Zhang, Xiangyang City (CN); Ting Yang, Beijing City (CN); HongTao Niu, Beijing (CN); Ke Huang, Beijing (CN); JianMei Jin, Shanghai City (CN)

(21) Appl. No.: 17/471,137

(22) Filed: Sep. 9, 2021

(30) Foreign Application Priority Data
Aug. 27, 2021 (CN) 202110992451.8

(51) Int. Cl. A61M 15/00

(52) U.S. Cl. CPC A61M 15/0001; A61M 15/0065; A61M 2205/8206

(57) ABSTRACT

The present invention relates to the technical field of an automatic detection trigger device, and discloses an automatic detection trigger device for a quantitative compressed administration aerosol. The automatic detection trigger device for the quantitative compressed administration aerosol includes a main machine body, wherein the main machine body consists of a main machine front cover, a main machine rear cover, a battery module and a driving assembly; the driving assembly includes a driving control module and a driving gear module; a charging port is formed at an outer side of the main machine front cover; a limiting pedestal is clamped at a bottom end of the main machine front cover; and an aerosol medicine is fixedly mounted at the bottom of an inner cavity of the main machine front cover. The automatic detection trigger device for the quantitative compressed administration aerosol detects an inspiration flow rate of a user in a medicine inhaling process through a flow sensor arranged in the driving assembly. When the inspiration flow rate reaches an ideal threshold, the device drives the aerosol to spray a medicine, and the medicine sprayed by the aerosol enters a lower respiratory tract such as a lung along with airflow, so that the problem of hand-mouth synchronous operation is solved, and the utilization rate of the medicine is greatly increased.

### TECHNICAL FIELD

The present invention relates to the technical field of an automatic detection trigger device, and in particular to an automatic detection trigger device for a quantitative compressed administration aerosol.

### BACKGROUND

Inhalation aerosol refers to a preparation as follows: a medicine-containing solution, suspension or emulsion, and an appropriate propellant or a liquefied mixed propellant are jointly packaged in a pressure-resistant container with a quantitative valve system and a certain pressure; and during use, the content is sprayed out as mist by means of the pressure of the propellant, and then is inhaled through a mouth and deposited in a lung. Generally, the inhalation aerosol is also called a pressure quantitative inhalant. Active substances can be quantitatively released by pressing the valve, and the medicine is dispersed into particles or frog drops which are inhaled through the respiratory tract to play a local or systemic treatment role.

The pressure quantitative inhalant, which came out in the mid-1950s, is currently the most widely used administration device. It has the advantages of small size, portability, rapid action, convenience in use, low price, multi-dose administration and the like. The structure consists of two parts: a plastic fixed base (including a suction port) and a quantitative valve. When the medicine liquid is driven by a user to manually lift and press the valve, the medicine and the propellant are sprayed out in the form of aerosol. Due to the unique design structure, the pressure quantitative aerosol inhaler can ensure that the amount of solution ejected by manual pressing is within a certain range. In order to achieve the best inhalation treatment effect, the pressure quantitative inhalant requires the user to slowly inhale deeply while manually lifting and pressing so as to inhale the medicine mist into the respiratory tract and lung. However, it is difficult for some people to ensure hand-mouth synchronization, resulting in increased deposition amount of the medicine mist in the oropharynx, low deposition rate in lung and low medicine utilization rate.

### SUMMARY

### (I) Technical Problem to be Solved

In view of the shortcomings of the, prior art, the present invention provides an automatic detection trigger device for a quantitative compressed administration aerosol, so as to solve the problems in the background art.

### (II) Technical Solutions

In order to solve the above objective, the present invention provides the following technical solution: an automatic detection trigger device for a quantitative compressed administration aerosol includes a main machine body, wherein the main machine body consists of a main machine front cover, a main machine rear cover, a battery module and a driving module; the driving assembly includes a driving control module and a driving gear module;

the driving control module includes a driving module bracket, a display screen, a control button, a control mainboard, a driving motor, a trigger detection button, a trigger key baffle, an air inlet hole, an aerosol holder, a flow sensor and a reset spring;

the driving gear module includes a small helical gear, a first speed-reducing gear, a second speed-reducing gem, a rotary cam, a push plate and a push column;

a charging port is formed at an outer side of the main machine front cover; a limiting pedestal is clamped at a bottom end of the main machine front cover; and an aerosol medicine is fixedly mounted at the bottom of an inner cavity of the main machine front cover.

Preferably, the main machine rear cover is clamped on one side of the main machine front cover, the battery module is clamped at the main machine front cover and located below the main machine rear cover, and the driving assembly is fixedly mounted at the top of the inner cavity of the main machine front cover.

Preferably, the display screen is fixedly mounted on one side of a front surface of the driving module bracket, the control button is fixedly mounted on the front surface of the driving module bracket and located below the display screen, the control mainboard is fixedly connected to one side of the driving module bracket, and the driving motor is fixedly mounted on one side of the driving module bracket and located below the control mainboard.

Preferably, the trigger detection button is fixedly mounted at the top of the other side of the driving module bracket the trigger key baffle is mounted below the trigger detection button, the, air inlet hole is formed at the bottom of the other side of the driving module bracket, the aerosol holder is fixedly connected to a bottom end of the driving module bracket, the flow sensor is fixedly mounted on one side of an inner wall of the aerosol holder, and the reset spring is fixedly connected to a top end of the aerosol holder.

Preferably, the small helical gear is in fixed sleeving connection with an outer side of an output shaft of the driving motor, the first speed-reducing gear is engaged with a top end of the small helical gear, the second speed-reducing gear is engaged with one side of the first speed-reducing gear, the rotary camp is fixedly connected to one side of the second speed-reducing gear, the push plate is fixedly connected to a bottom end of the rotary cam, and the push column is fixedly connected to a bottom end of the push plate.

Preferably, a bottom end of the push column penetrates through the aerosol holder and is attached to a top end of the aerosol medicine, the reset spring is in sleeving connection with an outer side of the push column, and a top end of the reset spring is fixedly connected to the bottom end of the push plate.

### (III) Beneficial Effects

Compared with the prior art, the present invention provides an automatic detection trigger device for a quantitative compressed administration aerosol, and has the following beneficial effects:

the automatic detection trigger device for the quantitative compressed administration aerosol may detect an inspiration flow rate of a user in a medicine inhaling process through a flow sensor arranged in the driving assembly. When the inspiration flow rate reaches an ideal threshold, the device drives the aerosol to spray a medicine, and the medicine sprayed by the aerosol enters a lower respiratory tract such as a lung along with airflow, so that the problem of hand-mouth synchronous operation is solved, and the utilization rate of the medicine is greatly increased.

### DESCRIPTION OF THE DRAWINGS

FIG. 1 is a structural schematic diagram of the present invention;

FIG. 2 is a structural schematic diagram after assembling of the present Invention;

FIG. 3 is a side view after assembling of the present Invention;

FIG. 4 is a structural schematic diagram of a driving assembly according to the present invention;

FIG. 5 is a bottom view of a driving assembly according to the present invention;

FIG. 6 is a structural schematic diagram of a driving gear module according to the present invention;

FIG. 7 is a bottom view of a driving gear nodule according to the present invention; and

FIG. 8 is an electrical working diagram of the present invention.

In the drawings: 1. Main, machine body; 101. Main machine front cover; 102. Main machine rear cover; 2. Charging port; 3. Limiting pedestal; 4. Battery module; 5. Aerosol medicine; 67. Driving assembly; 61. Driving control module; 6101. Driving module bracket; 6102. Display screen; 6103. Control button; 6104. Control mainboard; 6105. Driving motor; 6106. Trigger detection button; 6107. Trigger key baffle; 6108. Air inlet hole; 6109. Aerosol holder; 6110. Flow sensor; 6111. Reset spring; 71. Driving gear module; 7101. Small helical gear; 7102. First speed-reducing gear; 7103. Second speed-reducing gear; 7104. Rotary camp; 7105. push plate; 7106. Push column.

### DETAILED DESCRIPTION OF EMBODIMENTS

The following clearly and completely describes the technical solutions in the embodiments of the present invention with reference to accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

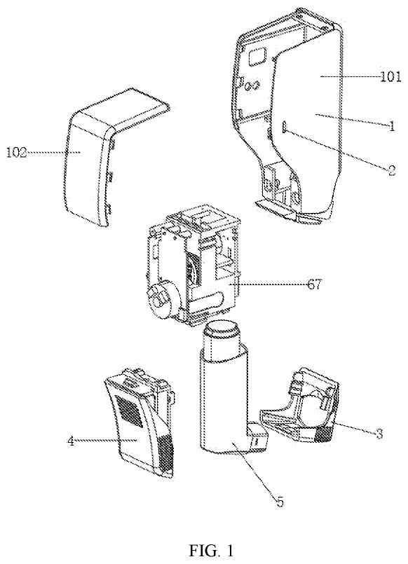

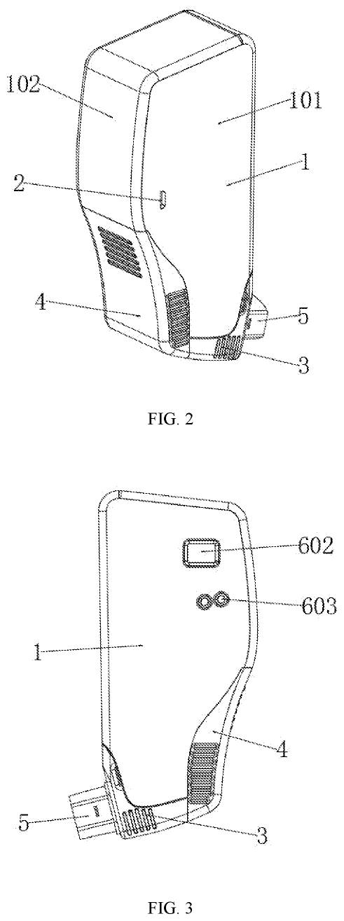

The present invention provides a technical solution: an automatic detection trigger device for a quantitative compressed administration aerosol includes a main machine body 1. Referring to FIG. 1, the main, machine body 1 consists of a main machine front cover 101, a main machine rear cover 102, a battery module 4 and a driving assembly 67. Referring to FIG. 2 and FIG. 3, the main machine rear cover 102 is clamped on one side of the main machine front cover 101, the battery module 4 is clamped at the main machine front cover 101 and located below the main machine rear cover 102, the battery module 4 is located at a rear end of the main machine and is used to supply electric energy required for operation of the device, the battery module 4 adopts a chargeable lithium battery, the driving assembly 67 is fixedly mounted at the top of an inner cavity of the main machine front cover 101, a charging port 2 is formed on an outer side of the main machine front cover 101, the charging port 2 is applied to an external USB cable to charge device, a limiting, pedestal 3 is clamped at the bottom end of the main machine front cover 101, an aerosol medicine 5 is fixedly mounted at the bottom of the inner cavity of the main machine front cover 101, a medicine detection sensor is mounted outside the aerosol medicine 5, and the medicine detection sensor is used to detect whether the aerosol medicine is inserted in the device. After the aerosol medicine 5 is inserted, the medicine detection sensor may detect a medicine tank so as to start a corresponding function. The common medicines in the market are stored in the aerosol medicine 5. The limiting pedestal 3 is located at the bottom of the main machine body 1 and is used to fix the aerosol medicine 5.

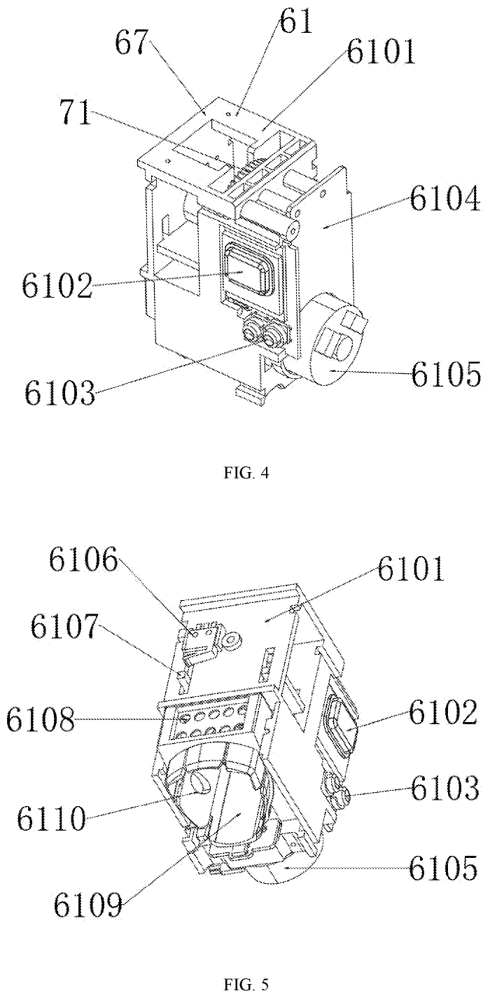

Referring to FIG. 4 and FIG. 5, the driving assembly 67 includes a driving control module 61 and a driving gear module 71, wherein the driving control module 61 includes a driving module bracket 6101, a display screen 6102, a control button 6103, a control mainboard 6104, a driving motor 6105, a trigger detection button 6106, a trigger key baffle 6107, an air inlet hole 6108, an aerosol holder 6109, a flow sensor 6110 and a reset spring 6111; and the driving module bracket 6101 is used to drive the gear module 71 to be fixed with other parts and is connected into a whole body through all the parts.

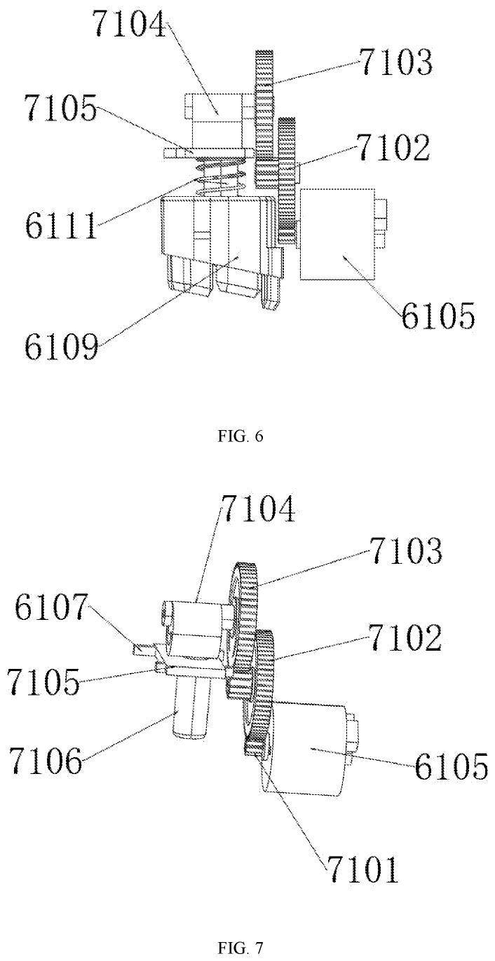

The display screen 6102 is fixedly mounted on one side of a front surface of the driving module bracket 6101, and the display screen 6102 is used to display device state information and medicine suction detection data; a user may intuitively view related information through the screen; the control button 6103 is fixedly mounted on the front surface of the driving module bracket 6101 and located below the display screen 6102, and the control button 6103 is used to control and set functions of the device; the control mainboard 6104 is fixedly connected to one side of the driving module bracket 6101, and the control mainboard 6104 is used to control various functions of the device; the driving motor 6105 is fixedly mounted on one side of the driving module bracket 6101 and located below the control mainboard 6104, and the driving motor 6105 is electrically connected to the battery module 4 through an external power line; the trigger detection button 6106 is fixedly mounted at the top of the other side of the driving module bracket 6101; the trigger key baffle 6107 is mounted below the trigger detection button 6106; the air inlet hole 6108 is formed at the bottom of the other side of the driving module bracket 6101; the aerosol holder 6109 is fixedly connected to the bottom end of the driving module bracket 6101, and the aerosol holder 6109 is located at the lower end of the driving gear module 71 and used to position the aerosol medicine 5 and the device; the flow sensor 6110 is fixedly mounted on one side of an inner wall of the aerosol holder 6109, and the flow sensor 6110 is used to detect whether the aerosol medicine 5 is inserted into the device; after the aerosol medicine 5 is inserted, the flow sensor 6110 may detect a medicine tank so as to start a corresponding function; the reset spring 6111 is fixedly connected to the top end of the aerosol holder 6109; and referring to FIG. 6, at this time, the reset spring 6111 is in an unstressed state.

Referring to FIG. 6 and FIG. 7, the driving gear module 71 includes a small helical gear 7101, a first speed-reducing gear 7102, a second speed-reducing gear 7103, a rotary cam 7104, a push plate 7105 and a push column 7106; the driving gear module 71 is a moment conduction device and performs step-by-step transfer and phase change on the moment of the driving motor 6105; the small helical gear 7101 is in fixed sleeving connection with an outer side of an output shaft of the driving motor 6105; the first speed-reducing gear 7102 is engaged with the top end of the small helical gear 7101; the second speed-reducing gear 7103 is engaged with one side of the first speed-reducing gear 7102; the rotary cam 7104 is fixedly connected to one side of the second speed-reducing gear 7103 the push plate 7105 is fixedly connected to the bottom end of the rotary cam 7104; the push column 7106 is fixedly connected to the bottom end of the push plate 7105; the bottom end of the push column 7106 penetrates through the aerosol holder 6109 and is attached to the top end of the aerosol medicine 5; the reset spring 6111 is in sleeving connection with an outer side of the push column 7106; and the top end of the reset spring 6111 is fixedly connected to the bottom end of the push plate 7105.

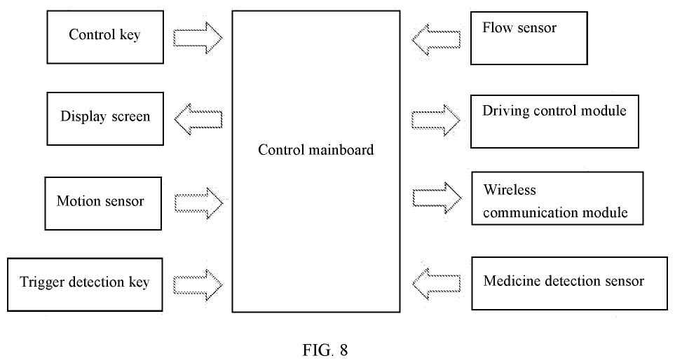

Referring to FIG. 8, when the medicine detection sensor detects that the medicine is filled in the device, the device is activated and starts to work. A motion sensor is used to detect the action and posture of medicines. Some medicines need to be shaken uniformly before use and need to be used in an erected posture. The motion sensor may detect the data so as remind the user of some unqualified use methods. The flow sensor 6110 is used to detect the flow of the inhaled gas. The control mainboard 6104 can trigger the driving control module 61 only when the detected flow continuously reaches an effective inhalation rate. A proper flow rate has a great influence on the deposition rate of the medicine, and too high or too low deposition rate will affect the absorption efficiency of the medicine. The flow sensor 6110 may detect a complete inhalation process and analyze whether the inhalation duration and the inhalation strength are reasonable, thereby giving more effective inhalation suggestions, increasing the inhalation rate of the medicine and achieving a better treatment effect.

The trigger detection button 6106 is used to detect the state of the driving control module 61 during work and feed a signal back to the control mainboard 6104. The control button 6103 is used to control information entry and function of the device, for example, resetting medicine dosage and turning pages to view data. A wireless communication module is a long-distance communication mode of the device, and the control mainboard 6104 acquires and records the device state information and the use process information, and may transmit the information to a mobile phone, a computer and a cloud through a wireless communication mode, thereby facilitating, data view and data analysis. The wireless communication nodule may be a communication mode such as Bluetooth, WIFI, a mobile information system and other wireless communication technologies.

The working principle of the device is as follows: during use, the driving motor 6105 rotates to drive the small helical gear 7101 to rotate, the small helical gear 7101 is engaged with the first speed-reducing gear 7102, the first speed-reducing gear 7102 is engaged with the second speed-reducing gear 7103, the rotary cam 7104 is fixedly on the second speed-reducing gear 7103 and rotates with the second speed-reducing gear, and a rotary moment of the driving motor 6105 is increased step by step after passing through the first speed-reducing gear 7102 and the second speed-reducing gear 7103, so that a force capable of driving the aerosol to spray the medicine is formed.

The rotary cam 7104 rotates to push the push plate 7105, the trigger key baffle 6107 is arranged on the push plate 7105, the push column 7106 is arranged below the push plate 7105, the push column 7106 is in contact with the top of the aerosol medicine 5, and the push plate 7105 moves up and down to push the push column 7106 to move up and down so as to push the aerosol medicine 5 for pressing and spraying.

When a user inhales the medicine and the control mainboard 6104 detects that the flow reaches to a proper flow, the driving assembly 67 is controlled to work, the push column 7106 pushes the medicine to complete one-time spraying, and the medicine spray enters the lower respiratory tract of a human body along with inhalation. After the push column 7106 pushes the medicine, the trigger key baffle 6107 triggers the trigger detection button 6106 and feeds a feedback signal back to the control mainboard 6104, and the control mainboard 6104 controls the driving assembly 67 to stop work, so that the medicine is automatically detected for one time.

It should be noted that in this specification, relational terms such as first and second are used only to differentiate an entity or operation from another entity or operation, and do not require or imply that any actual relation or sequence exists between these entities or operations. In addition, the terms “comprise”, “include” and any other variants thereof are intended to cover non-exclusive inclusion, so that a process, a method, an article, or a device that includes a series of elements not only includes these very elements, but may also include other elements not expressly listed, or also include elements inherent to this process, method, article, or device.

Although the embodiments of the present invention have been illustrated and described, it should be understood that those of ordinary skill in the art may make various changes, modifications, replacements and variations to the above embodiments without departing from the principle and spirit of the present invention, and the scope of the present invention is limited by the appended claims and their legal equivalents.

1. An automatic detection trigger device for a quantitative compressed administration aerosol, comprising a main machine body 1, wherein the main machine body consists of a main machine front cover, a main machine rear cover, a battery module and a driving module; the driving assembly comprises a driving control module and a driving gear module;
the driving control module comprises a driving module bracket, a display screen, a control button, a control mainboard, a driving motor, a trigger detection button, a trigger key baffle, an air inlet hole, an aerosol holder, a flow sensor and a reset spring;
the driving gear module comprises a small helical gear, a first speed-reducing gear, a second speed-reducing gear, a rotary cam, a push plate and a push column;
a charging port is formed at an outer side of the main machine front cover; a limiting pedestal is clamped at a bottom end of the main machine front cover; and an aerosol medicine is fixedly mounted at the bottom of an inner cavity of the main machine front cover.

2. The automatic detection trigger device for the quantitative compressed administration aerosol according to claim 1, wherein the main machine rear cover is clamped on one side of the main machine front cover, the battery module is clamped at the main machine front cover and located below the main machine rear cover, and the driving assembly is fixedly mounted at the top of the inner cavity of the main machine front cover.

3. The automatic detection trigger device for the quantitative compressed administration aerosol according to claim 1, wherein the display screen is fixedly mounted on one side of a front surface of the driving module bracket, the control button is fixedly mounted on the front surface of the driving module bracket and located below the display screen, the control mainboard is fixedly connected to one side of the driving module bracket, and the driving motor is fixedly mounted on one side of the driving module bracket and located below the control mainboard.

4. The automatic detection trigger device for the quantitative compressed administration aerosol according to claim 1, wherein the trigger detection button is fixedly mounted at the top of the other side of the driving module bracket, the trigger key baffle is mounted below the trigger detection button, the air inlet hole is formed at the bottom of the other side of the driving module bracket, the aerosol holder is fixedly connected to a bottom end of the driving module bracket, the flow sensor is fixedly mounted on one side of an inner wall of the aerosol holder, and the reset spring is fixedly connected to a top end of the aerosol holder.

5. The automatic detection trigger device for the quantitative compressed administration aerosol according to claim 1, wherein the small helical gear is in fixed sleeving connection with an outer side of an output shaft of the driving motor, the first speed-reducing gear is engaged with a top end of the small helical gear, the second speed-reducing gear is engaged with one side of the first speed-reducing gear, the rotary camp is fixedly connected to one side of the second speed-reducing gear, the push plate is fixedly connected to a bottom end of the rotary cam, and the push column is fixedly connected to a bottom end of the push plate.

6. The automatic detection trigger device for the quantitative compressed administration aerosol according to claim 1, wherein a bottom end of the push column penetrates through the aerosol holder and is attached to a top end of the aerosol medicine, the reset spring is in sleeving connection with an outer side of the push column, and a top end of the reset spring is fixedly connected to the bottom end of the push plate.
